SEQUENCE LISTING (24 sequences)

SEQ ID NO: 1 - DNA, 21 bp, Artificial Sequence
  Inventor Uematsu, Chihiro ; Okano Kazunori
aagggcgtaaccgaaatcggt

SEQ ID NO: 2 - DNA, 65 bp, Artificial Sequence
  Description of Artificial Sequence reverse DNA primer which is used in NASBA reaction and hybridizes with Human Papillomavirus DNA
aattctaatacgactcactatagggcccttctcactgttctctcatgtttgcagctctgtgcata

SEQ ID NO: 3 - DNA, 21 bp, Artificial Sequence
  Description of Artificial Sequence DNA probe which is used in real-time detection of amplified fragments
cccttctcactgttctctcat

SEQ ID NO: 4 - DNA, 18 bp, Artificial Sequence
  Description of Artificial Sequence forward DNA primer which is used in NASBA reaction and hybridizes with Human Insulin Gene
tggtgcaggcagcctgca

SEQ ID NO: 5 - DNA, 66 bp, Artificial Sequence
  Description of Artificial Sequence reverse DNA primer which is used in NASBA reaction and hybridizes with Human Insulin Gene
aattctaatacgactcactatagggcccttctcactgttctctcattagttgcagtagttctccag

SEQ ID NO: 6 - DNA, 19 bp, Artificial Sequence
  Description of Artificial Sequence forward DNA primer which is used in NASBA reaction and hybridizes with Human Insulin Gene
ccagccgcagcctttgtga

SEQ ID NO: 7 - DNA, 65 bp, Artificial Sequence
  Description of Artificial Sequence reverse DNA primer which is used in NASBA reaction and hybridizes with Human Insulin Gene
aattctaatacgactcactatagggcactcatctcttctccctgttcaggtcctctgcctcccgg

SEQ ID NO: 8 - DNA, 21 bp, Artificial Sequence
  Description of Artificial Sequence DNA/RNA chimera probe which is used in real-time detection of amplified fragments
cccttctcacuguuctctcat

SEQ ID NO: 9 - DNA, 21 bp, Artificial Sequence
  Description of Artificial Sequence DNA/RNA chimera probe which is used in real-time detection of amplified fragments
cactcatcucuucuccctgtt

SEQ ID NO: 10 - DNA, 20 bp, Artificial Sequence
  Description of Artificial Sequence forward DNA primer which is used in NASBA reaction and hybridizes with core gene of hepatitis C virus genotype I/1a
ggtcgcaacgtcgaggtaga

SEQ ID NO: 11 - DNA, 20 bp, Artificial Sequence
  Description of Artificial Sequence forward DNA primer which is used in NASBA reaction and hybridizes with core gene of hepatitis C virus genotype II/1b
cgcaacctcgtggaaggcga

SEQ ID NO: 12 - DNA, 20 bp, Artificial Sequence
  Description of Artificial Sequence forward DNA primer which is used in NASBA reaction and hybridizes with core gene of hepatitis C virus genotype III/2a
ccccccgaggttcccgtgcc

SEQ ID NO: 13 - DNA, 20 bp, Artificial Sequence
  Description of Artificial Sequence forward DNA primer which is used in NASBA reaction and hybridizes with core gene of hepatitis C virus genotype IV/2b
ctgtacggaaacgagggttg

SEQ ID NO: 14 - DNA, 20 bp, Artificial Sequence
  Description of Artificial Sequence forward DNA primer which is used in NASBA reaction and hybridizes with core gene of hepatitis C virus genotype V/3a
cgacgcgtaaaacttctcaa

SEQ ID NO: 15 - DNA, 66 bp, Artificial Sequence
  Description of Artificial Sequence reverse DNA primer which is used in NASBA reaction and hybridizes with core gene of hepatitis C virus genotype I/1a
aattctaatacgactcactatagggcccttctcactgttctctcatgagccatcccgcccaccagc

SEQ ID NO: 16 - DNA, 66 bp, Artificial Sequence
  Description of Artificial Sequence reverse DNA primer which is used in NASBA reaction and hybridizes with core gene of hepatitis C virus genotype II/1b
aattctaatacgactcactatagggcactcatctcttctccctgttgagccatcctgyccacgcya

SEQ ID NO: 17 - DNA, 66 bp, Artificial Sequence
  Description of Artificial Sequence reverse DNA primer which is used in NASBA reaction and hybridizes with core gene of hepatitis C virus genotype III/2a
aattctaatacgactcactatagggctctgttccctcatcacttctccttacccacgttgcgctac

SEQ ID NO: 18 - DNA, 66 bp, Artificial Sequence
  Description of Artificial Sequence reverse DNA primer which is used in NASBA reaction and hybridizes with core gene of hepatitis C virus genotype IV/2b
aattctaatacgactcactatagggcccttctctctcatcactgttggtcggtggggccccaatta

SEQ ID NO: 19 - DNA, 66 bp, Artificial Sequence
  Description of Artificial Sequence reverse DNA primer which is used in NASBA reaction and hybridizes with core gene of hepatitis C virus genotype V/3a
aattctaatacgactcactatagggcactcatccctgttctcttctaggaccggccttcgctccga

SEQ ID NO: 20 - DNA, 21 bp, Artificial Sequence
  Description of Artificial Sequence DNA probe which is used in detection of amplified fragments
cccttctcactgttctctcat

SEQ ID NO: 21 - DNA, 21 bp, Artificial Sequence
  Description of Artificial Sequence DNA probe which is used in detection of amplified fragments
cactcatctcttctccctgtt

SEQ ID NO: 22 - DNA, 21 bp, Artificial Sequence
  Description of Artificial Sequence DNA probe which is used in detection of amplified fragments
ctctgttccctcatcacttct

SEQ ID NO: 23 - DNA, 21 bp, Artificial Sequence
  Description of Artificial Sequence DNA probe which is used in detection of amplified fragments
cccttctctctcatcactgtt

SEQ ID NO: 24 - DNA, 21 bp, Artificial Sequence
  Description of Artificial Sequence DNA probe which is used in detection of amplified fragments
cactcatccctgttctcttct

(19) United States
(12) Patent Application Publication
(10) Pub. No.: US 2004/0209291 A1
(43) Pub. Date: Oct. 21, 2004

(54) Method for expressed gene analysis and probe kit for expressed gene analysis

(76) Inventors: Chihiro Uematsu, Kawasaki (JP); Kazunori Okano, Shiki (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/759,179

(22) Filed: Jan. 20, 2004

(30) Foreign Application Priority Data
Apr. 18, 2003 (JP) 2003-114721

(51) Int. Cl. C12Q 1/68; C12P 19/34

(52) U.S. Cl. 435/6; 435/91.2

(57) ABSTRACT

This invention provides a novel kit for detecting nucleic acid that can be universally used independent of the target nucleic acid sequence, and a simple method for detecting nucleic acid utilizing the same. This method comprises: subjecting a gene to be analyzed to real-time detection using a primer comprising a base sequence specifically hybridizing to the target gene or nucleic acid and the TaqMan® probe or the Molecular Beacon comprising a base sequence identical or complementary to the first base sequence, wherein the gene to be analyzed is prepared by introducing the first base sequence and the second base sequence comprising the T7 promoter sequence, which are nonspecific to the base sequence of the target gene or nucleic acid, into the target gene or nucleic acid so that the second base sequence is bound to a position closer to the 5′ end than the first base sequence. This invention also provides a universal probe for detecting nucleic acid. The use of the two types of universal probes of the present invention enables simultaneous real-time detection of several target genes in a single reaction vessel.

This patent application claims priority from Japanese Patent Application No. 2003-114721 and includes part or all of the contents as disclosed in the description thereof.

### TECHNICAL FIELD

The present invention relates to a method for expressed gene analysis and a probe kit for expressed gene analysis through fluorescent energy transfer. More particularly, the present invention relates to a method for expressed gene analysis and a probe kit for expressed gene analysis using a probe that can be universally used regardless of the target gene sequence, which is realized by the introduction of a sequence independent of the base sequence of the target gene therein.

### BACKGROUND ART

In the past, reverse transcription-polymerase chain reaction (RT-PCR) was employed for detection of RNA. In RT-PCR, cDNA is first synthesized from a target gene by reverse transcription and is then subjected to amplification by PCR, and the PCR product is then detected. Nucleic acid sequence-based amplification (NASBA®) is known as a method for amplifying the target gene to at least 10¹²-fold of the original amount in a reaction using reverse transcriptase, ribonuclease H, and T7 RNA polymerase for 90 to 120 minutes (J. Compton: Nucleic acid sequence-based amplification, Nature, 1991, 350, pp. 91-92). Since NASBA® reaction is carried out under isothermal conditions at 41° C., genomic DNAs other than the target gene can be prevented from being heat-denatured. Accordingly, the target gene can be more specifically amplified through this reaction than through RT-PCR. Unlike PCR, this reaction does not require any thermal cycle. Thus, the reaction conditions do not need to be modified in accordance with the target gene sequence. This enables amplification with an apparatus of a simple structure.

When assaying the amount of PCR products, fluorescent detection and electrophoresis were often employed in combination. In general, the size of the amplified product was first confirmed by electrophoresis, the fluorescent intensity was assayed, and the amount of the amplified product was then assayed. Recently, several methods that would enable real-time fluorescent detection without electrophoresis the PCR product have been reported (Pamela M. et al., Proc. Natl. Acad. Sci., USA, August 1991, Vol. 88, pp. 7276-7280; S Tyagi, F R Kramer, Nature Biotechnology, 1996, Vol. 14, pp. 303-308). In these methods, a probe that is devised to emit fluorescence by hybridizing to the PCR product through fluorescent energy transfer (for example, a TaqMan® probe or the Molecular beacon) is used for real-time detection. During experiment, the PCR products can be assayed for each cycle, and thus, a region of PCR cycles in which the PCR products are exponentially amplified and a region of PCR cycles in which the PCR products have reached the plateau phase can be determined simply. Accordingly, labor in RT-PCR was reduced, and this method rapidly became widely used as an effective method for expressed gene analysis.

In a method for detecting PCR products through fluorescent energy transfer, however, a probe or primer for detecting the target gene must be individually designed for each target gene. In addition, such a probe is disadvantageously cost-intensive due to the use of fluorescent energy transfer and has difficulty that design guidelines are different from those of general primers.

A method for nucleic acid amplification utilizing the Molecular beacon that can be universally used regardless of the target gene sequence was also developed (U.S. Pat. No. 6,090,552). In this method, NASBA® can be employed for gene amplification.

In contrast, Whitcombe et al. have reported fluorescent assay for PCR products utilizing the TaqMan® probe that can be universally used regardless of the target gene sequence (Whitcombe D. et al., Clinical Chemistry, 1998, Vol. 44, No. 5, pp. 918-923). This assay targets genomic DNA and aims at single nucleotide polymorphism (SNP) typing. In this method, a template-non-specific probe sequence and a Tag sequence are introduced into genomic DNA. This enables assay using the template-non-specific TaqMan® probe. In this method, however, two types of primer pairs are used, i.e., a primer pair for introduction and another primer pair hybridizing to the Tag sequence of the synthesized DNA to amplify the DNA. This necessitates two different thermal cycles, and results in the unavoidable production of by-products. When real-time detection is carried out in a single reaction vessel, reaction properties of the two types of probes cannot be precisely controlled at the same level due to the difference in Tm values of the probes. Accordingly, this assay is not convenient for the quantitative analysis of gene expression.

Objects of the present invention are to solve the problems of the prior art and to provide a novel kit for expressed gene analysis that can be universally used regardless of the target gene sequence, as well as a simple method for expressed gene analysis utilizing the same.

### DISCLOSURE OF THE INVENTION

In order to attain the above objects, the present invention provides the following method for expressed gene analysis:

a method for expressed gene analysis comprising: subjecting a gene to be analyzed to nucleic acid amplification using a forward primer specifically hybridizing to the gene to be analyzed, a primer for introduction comprising a first base sequence closer to the 5′ end than a third base sequence comprising a sequence specifically hybridizing to a target gene and comprising a second base sequence closer to the 5′ end than the first base sequence, a probe comprising a base sequence identical or complementary to the first base sequence and labeled at one end with a fluorophore and at another end with a quencher, reverse transcriptase, RNA polymerase, and ribonuclease H and/or exonuclease;

digesting the probe bound to the first base sequence by the ribonuclease H or exonuclease at the time of the nucleic acid amplification; and

detecting fluorescence emitted by the released fluorophore, thereby assaying the amount of the product of the nucleic acid amplification,

wherein the gene to be analyzed is prepared by introducing the first base sequence and the second base sequence comprising a promoter sequence of RNA polymerase, which are nonspecific to the base sequence of the target gene, into the target gene so that the second base sequence is bound to a position closer to the 5′ end than the first base sequence.

The first base sequence and the second base sequence are introduced into the target gene using a primer for introduction, which is constituted by three sequence portions. This primer for introduction comprises the first base sequence, which is closer to the 5′ end than the third base sequence comprising a sequence specifically hybridizing to the target gene, and the second base sequence, which is closer to the 5′ end than the first base sequence. The first portion is a sequence identical or complementary to the probe for detection, and the second portion is a sequence comprising the promoter sequence and the transcription initiation site of RNA polymerase. The first, second, and third portions may be continuous, or there may be a joining portion between two portions. Although the third sequence varies depending on the target gene sequence, the first sequence can be freely designed independent of the target gene sequence. The second sequence is a constant sequence independent of the target gene sequence (e.g., T7 promoter sequence).

The aforementioned forward primer is one for amplifying the gene to be analyzed or a part thereof. It may be any oligonucleotide hybridizing to a position closer to the 3′ end than the third portion that had been introduced to the gene to be analyzed.

The RNA polymerase that is used in the present invention is not particularly limited. Any RNA polymerase, such as T7 RNA polymerase, T3 RNA polymerase, or SP6 RNA polymerase, can be used, with T7 RNA polymerase being preferable. When T7 RNA polymerase is used, the aforementioned second portion preferably comprises the T7 promoter sequence.

For example, when mRNA of a certain target gene is to be detected, a primer for introduction that comprises the aforementioned three sequence portions is first used as a reverse transcription primer to synthesize first strand cDNA from target RNA (mRNA). This cDNA functions as a gene to be analyzed (single-stranded CDNA) prepared by introducing the first and the second base sequences (the promoter sequences of RNA polymerase) to the target gene. Subsequently, the obtained gene to be analyzed (cDNA) is used as a template to synthesize second strand cDNA. Thus, a gene to be analyzed (double-stranded cDNA) having the promoter sequence of RNA polymerase is synthesized.

The synthesized gene to be analyzed that comprises a promoter sequence of the RNA polymerase is amplified in the following manner.

1) First, the gene to be analyzed is transcribed into RNA with the aid of RNA polymerase. The RNA polymerase recognizes a promoter sequence of the gene to be analyzed and transcribes many cRNAs (antisense RNAs) of mRNA of the target gene.

2) Subsequently, the aforementioned RNA is subjected to reverse transcription using the forward primer and a reverse transcriptase or ribonuclease H to synthesize single-stranded cDNA.

3) The gene to be analyzed is then synthesized from the single-stranded cDNA using the primer for introduction. Synthesis may be carried out by adding an additional DNA polymerase. Alternatively, the reverse transcriptase may be used since it generally has DNA polymerase activity.

The synthesized gene to be analyzed is used as a template in transcription in step 1), and steps 1) to 3) are sequentially repeated. Thus, amplification proceeds.

The thus amplified double-stranded cDNA is detected using the TaqMan® probe consisting of a sequence identical or complementary to the first base sequence. The TaqMan® probe is a DNA probe which is labeled at its 5′ end with a fluorophore and at its 3′ end with a substance that quenches the fluorescence emitted by the fluorophore through energy transfer (quencher). The TaqMan® probe does not emit fluorescence in a usual state because the 5′ end and the 3′ end are labeled with a fluorophore and a quencher, respectively. At the time of amplification, however, the probe hybridizes to the target sequence, and the probe is digested by ribonuclease H or exonuclease. Thus, a free fluorophore is generated, and fluorescence is emitted.

In the method for expressed gene analysis of the present invention, nucleic acid amplification after reverse transcription is carried out at a substantially single temperature (isothermal) using a pair of primers. The term “substantially single temperature” refers to the temperature, at which enzymes such as reverse transcriptase, ribonuclease H, RNA polymerase, and exonuclease simultaneously have enzyme activities. Specifically, it is about 35° C. to 55° C., and preferably about 40 to 42° C. At this temperature, reverse transcription, synthesis of double-stranded cDNA, transcription, and probe digestion simultaneously proceed. However, double-stranded DNA comprising several hundreds or more nucleotides such as genomic DNA are not generally denatured in this temperature range. Accordingly, genomic DNA other than the target gene is not amplified in the process for expressed gene analysis of the present invention. More specifically, by simply conducting incubation at certain temperature, the target gene can be specifically detected without generating any reaction by-product.

The probe for detection can be freely designed regardless of the target gene sequence. Thus, it can be universally used regardless of the type of the target gene.

According to the method for expressed gene analysis of the present invention, use of two or more types of probes enables the simultaneous analyses of two or more target genes derived from single or several specimens using a single reaction vessel for one target gene.

For example, a specimen and a standard sample can be simultaneously analyzed to compare the quantitative ratio of the target sample to the specimen. In such analysis, the Tm values of the aforementioned probes are preferably set at substantially the same levels to control the reaction property of each probe at the same levels, thereby conducting accurate expression analysis.

The present invention also provides a kit for expressed gene analysis. This kit comprises a probe comprising a sequence identical or complementary to a first base sequence, and labeled at one end with a fluorophore and at another end with a quencher,

wherein both the first base sequence and the second base sequence located closer to the 5′ end than the first base sequence are nonspecific to a base sequence of a target gene and are introduced into the target gene.

When the simultaneous detection of several target genes is intended, the aforementioned kit preferably comprises two or more types of probes, and the Tm values of those probes are substantially the same. Examples of two or more types of probes having substantially the same Tm values are probes which consist of several module sequences each consisting of 3 or 4 bases, where both terminal bases (a base at the 5′ end and a base at the 3′ end) of each module sequence are identical to each other, and each probe is constituted by rearranging module sequences having identical terminal bases.

### BRIEF DESCRIPTION OF DRAWINGS

FIG. 1 is a diagram showing the procedures of real-time gene detection by NASBA® using the universal probe of the present invention.

FIG. 2 is a diagram showing the sequence correlation between the universal probe of the present invention and the reverse transcription primer.

FIG. 3 is a diagram showing the principle that the universal probe of the present invention hybridizes to the target nucleic acid and is then hydrolyzed, thereby emitting fluorescence.

FIG. 4 is a diagram schematically showing the sequences of two types of universal probes.

FIG. 5 is a diagram showing the procedures of real-time gene detection by NASBA® using two types of universal probes.

FIG. 6 is a graph showing the result of real-time NASBA® detection using the universal probe of the present invention, wherein changes in fluorescence signal levels are shown in relation to the reaction time.

FIG. 7 is a diagram schematically showing the structure of the DNA/RNA chimera probe, which is an embodiment of the universal probe of the present invention.

FIG. 8 is a graph showing the results of detection by real-time NASBA® using two types of universal probes, wherein changes in fluorescence signal levels are shown in relation to the reaction time.

FIG. 9 is a diagram schematically showing a process of typing base sequences of viruses in a single reaction tube by the method of the present invention.

FIG. 10 is a graph showing the result of typing core genes of HCV viruses using 5 types of probes and showing the fluorescence intensity of each fluorophore.

### PREFERRED EMBODIMENTS OF THE INVENTION

The present invention is hereafter described in detail with reference to the drawings.

### 1. Design of Primer for Introduction

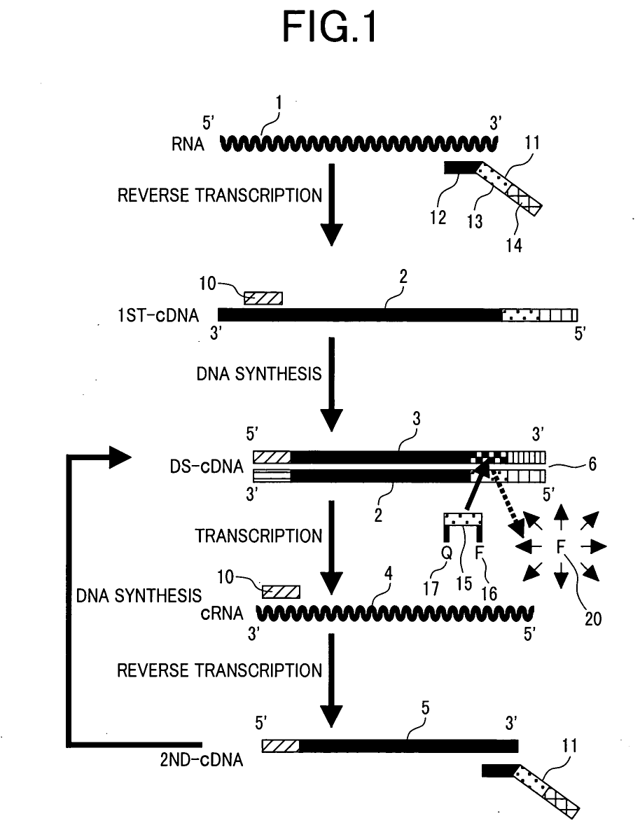

FIG. 1 is a diagram schematically showing the method for expressed gene analysis of the present invention, wherein the reference numeral 1 indicates a sample target RNA and the reference numeral 11 indicates a primer for introduction (a reverse transcription primer). The primer 11 for introduction is constituted by the sequence portion 12 hybridizing to the target RNA, the sequence portion 13 located closer to the 5′ end than the sequence portion 12 and consisting of a sequence identical to the probe for detection, and the sequence portion 14 comprising a T7 promoter sequence located closer to the 5′ end than the sequence portion 13.

The aforementioned sequence portions 12, 13, and 14 may be continuous, or there may be a joining portion between two portions. The length of the sequence portion 12 is not particularly limited, and it is preferably about 18 to 25 bases. The length of the sequence portion 13 is not particularly limited, and it is preferably about 18to 30 bases. The length of the sequence portion 14 is not particularly limited, and it is preferably about 20 to 25 bases. Although the sequence portion 12 varies depending on the target gene sequence, the sequence portion 13 can be freely designed independent of the target gene sequence. The sequence portion 14 is also independent of the target gene sequence and designed to comprise a promoter sequence or the transcription initiation site that are necessary for initiating the transcription of RNA polymerase. For example, when T7 RNA polymerase derived from T7 phage is used as the RNA polymerase, a sequence should be designed to comprise the T7 promoter sequence.

### 2. Synthesis of the Gene to be Analyzed (Reverse Transcription into cDNA)

The aforementioned primer for introduction is used as a reverse transcription primer, and cDNA is synthesized from mRNA comprising the target gene. Reverse transcription is carried out in accordance with a method known in the art. The aforementioned primer, the reverse transcriptase, and a substrate are added to a target RNA-containing reaction solution, and the mixture may be incubated at 35° C. to 45° C. for about 30 to 60 minutes. Any reverse transcriptase can be used without particular limitation, and an enzyme should also function as a DNA polymerase when synthesizing double-stranded cDNA from single-stranded cDNA. Examples thereof that can be used include M-MLV RT, AMV-RT, Omniscript®-RT (QIAGEN), and Sensiscript® (QIAGEN).

As a result of this reverse transcription, cDNA comprising the first base sequence 13 and the second base sequence 14, which are non-specific to the base sequence of the target gene and are introduced into the target gene, is synthesized as the gene to be analyzed (the first strand).

### 3. Design of Primer and Universal Probe

The design of a primer for amplifying the above-synthesized gene to be analyzed (cDNA) by NASBA® and that of a universal probe for expressed gene analysis are described.

As shown in FIG. 1, the forward primer 10 hybridizing to the gene to be analyzed and the primer 11 for introduction are used as the primers for amplification.

The probe 15, which has a sequence identical to the sequence portion 13 introduced into the gene to be analyzed, is used as a probe for detecting amplified products. The probe 15 is labeled with a fluorophore 16 indicated as “F” in the drawing and a quencher 17 indicated as “Q” in the drawing. In an intact state, fluorescence of the fluorophore 16 is eliminated through fluorescent energy transfer. However, the probe 15 releases the fluorophore and allows it to emit light with the progress of amplification. Examples of the fluorophore that can be used include fluorescein, tetrachlorofluorescein, hexachlorofluorescein, rhodamine, BODIPY, tetramethylrhodamine, Cy2, Cy3, Cy3B, Cy5, Cy7, Texas Red, ROX, FAM, and VIC. Examples of quenchers include 4-(4′-dimethylaminophenylazo)benzoic acid (DABCYL), Cy5Q, Cy7Q, NFQ, BHQ-0, BHQ-1, and BHQ-2. Among the aforementioned fluorophores, those that can quench fluorescence when it is brought into contact with another type of fluorophore can be used as a quencher.

The 3′ end of the probe 15 should be phosphorylated in order to prevent it from being elongated by reverse transcriptase or RNA polymerase.

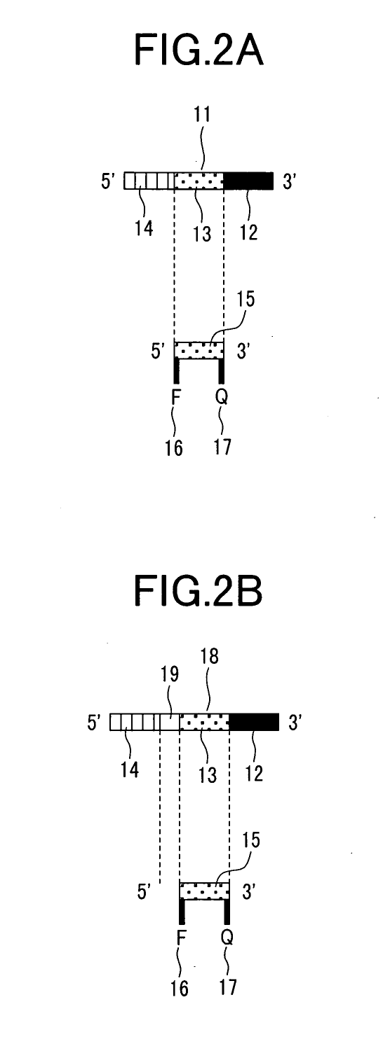

The sequence correlation between the primer 11 for introduction (the reverse transcription primer) and the probe 15 is described in more detail with reference to FIG. 2. In this drawing, the 5′ ends of the probe and the primer are on the left side and the 3′ ends thereof are on the right side.

As shown in FIG. 2(a), the primer 11 for introduction (the reverse transcription primer) is constituted by the sequence portion 12 hybridizing to target RNA, the sequence portion 13 consisting of a sequence identical to the probe 15, and the sequence portion 14 comprising a promoter sequence of RNA polymerase (e.g., a T7 promoter sequence). The sequence portion 14 is located closest to the 5′ end, the sequence portion 13 is located closer to the 3′ end than the sequence portion 14, and the sequence portion 12 is located closer to the 3′ end than the sequence portion 13. The probe 15 is labeled at its 5′ end with the fluorophore 16 and at its 3′ end with the quencher 17.

As shown in FIG. 2(b), there may exist a sequence portion 19, which is a joining portion between two sequence portions, in addition to the sequence portion 12 at which the primer 18 for introduction (the reverse transcription primer) hybridizes to target RNA, the sequence portion 13 consisting of a sequence identical to the probe 15, and the sequence portion 14 comprising a T7 promoter sequence. The length of the joining portion is not particularly limited, and it is preferably about 1 to 5 bases.

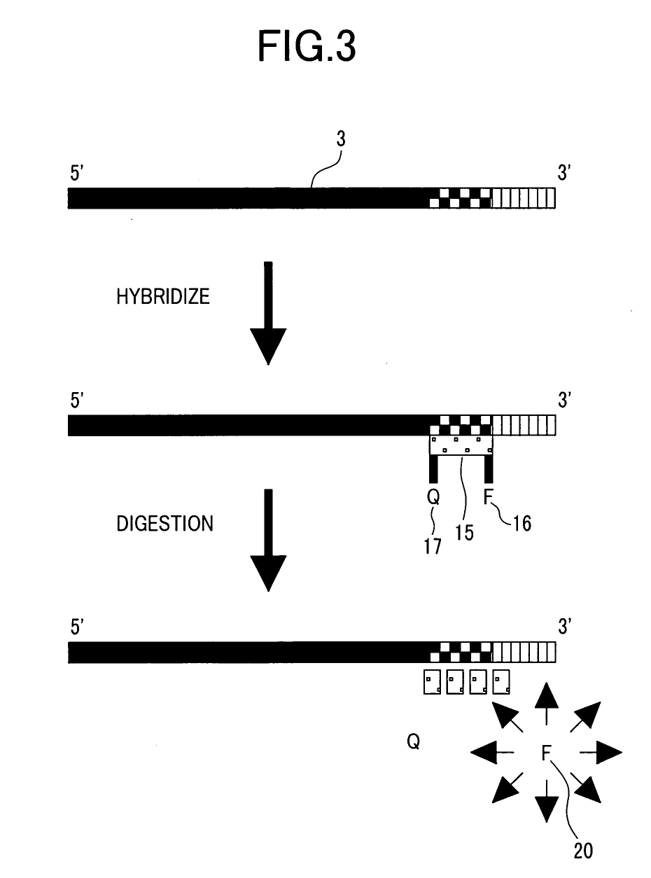

The mechanism of the probe 15 for emitting fluorescence is described with reference to FIG. 3. The probe 15 has to hybridize to DNA strand 3 that is synthesized during the amplification and has to be hydrolyzed by exonuclease or ribonuclease H in order to emit fluorescence. As a result of the hydrolysis, free fluorophore 20 is generated and fluorescence is emitted. The probe 15 that hybridized to the DNA strand 3 is digested by an enzyme having 5′→3′ exonuclease activity (e.g., T7 gene 6 exonuclease or Lamda exonuclease) from the 5′ end of the probe 15, digested by an enzyme having 3′→5′ exonuclease activity (e.g., exonuclease III) from the 3′ end, or digested by ribonuclease H having activity of specifically digesting only the RNA strand of a DNA/RNA hybrid strand. When ribonuclease H is used, therefore, the probe 15 should be an RNA probe having an RNA backbone or a probe having a DNA/RNA chimera backbone.

The sequence of the probe 15 can be designed independent of the sequence of target RNA. When assaying another target RNA, redesign of the sequence portion 12 of the reverse transcription primer 11 and the sequence of the forward primer 10 is sufficient, and the probe 15 can be used as a universal probe.

### 4. Amplification and fluorescent Emission

Amplification is carried out in accordance with a method known in the art by placing the gene to be analyzed, the forward primer, the primer for introduction, and the probe in a reaction tube and using reverse transcriptase, RNA polymerase, ribonuclease H, or exonuclease.

A process for amplification by NASBA® and that for fluorescent emission are described with reference to FIG. 1. At the outset, the primer 11 for introduction hybridizes to the gene 1 to be analyzed, reverse transcription proceeds, and first strand cDNA 2 is then synthesized. The first strand cDNA 2 comprises the first sequence portion 13 and the second sequence portion 14 introduced therein. The second sequence portion 14 comprises the T7 promoter sequence. Subsequently, the forward primer 10 hybridizes to first strand cDNA 2, second strand cDNA 3 is synthesized by DNA polymerase activity of reverse transcriptase, and double-stranded cDNA 6 having the T7 promoter sequence is generated.

Double-stranded cDNA has the T7 promoter sequence, and thus, many RNAs (cRNAs) 4 are transcribed by the T7 RNA polymerase. The forward primer 10 hybridizes to the transcribed RNA 4, reverse transcription proceeds, and cDNA 5 is synthesized. Further, the primer 11 for introduction hybridizes to cDNA 5, a DNA strand is synthesized, and double-stranded cDNA 6 is newly synthesized.

The probe 15 hybridizes to second strand cDNA 3 of the double-stranded cDNA 6. The probe 15 is hydrolyzed by exonuclease. As a result of the hydrolysis, fluorophore 16 is released from the probe 15, and free fluorophore 20 is generated, and fluorescence is emitted.

FIG. 1 shows a case where the probe 15 has a DNA backbone and is hydrolyzed by exonuclease. When the probe 15 has a DNA/RNA chimera backbone, detection can be similarly carried out. In this case, the probe 15 hybridizes to second strand cDNA 3, and the resulting RNA strand of the DNA/RNA hybrid is hydrolyzed by ribonuclease H.

FIG. 1 shows a case where a reverse transcription primer 11 has the first sequence portion 13 that is nonspecific to the target gene. Alternatively, the nonspecific first sequence portion 13 may be located at a position closer to the 5′ end of the forward primer 10.

Also, a Molecular Beacon probe may be used besides a TaqMan® probe. A primer 181 for introduction may comprise a sequence portion 182 (the third sequence portion) that hybridizes to the target RNA and a sequence portion 184 (the second sequence portion) that comprises an RNA polymerase promoter sequence. A forward primer 180 may comprise a sequence portion 179 (the third sequence portion) that hybridizes to the target RNA and a sequence portion 183 (the first sequence portion) that comprises a sequence identical to the probe sequence.

As shown in FIG. 11, the primer 181 for introduction is designed to comprise the second sequence portion 184 located at a position closer to the 5′ end than the third sequence portion 182. The forward primer 180 is designed to comprise the first sequence portion 183 located at a position closer to the 5′ end than the third sequence portion 179.

Processes of amplification and emission when the NASBA® is employed are described with reference to FIG. 11. At the outset, the primer 181 for introduction hybridizes to the gene 171 to be analyzed, reverse transcription proceeds, and whereby the first-strand cDNA 172 to which the second sequence portion 184 has been introduced is synthesized. Subsequently, the forward primer 180 hybridizes to the first-strand cDNA 172, and the second-strand cDNA 173 to which the first sequence portion 183 has been introduced is synthesized by DNA polymerase activity of a reverse transcriptase. As a result, a double-stranded cDNA 176 to which a second sequence portion 184 having a T7 promoter sequence and the first sequence portion 183 have been introduced is generated.

Since the double-stranded cDNA 176 has a T7 promoter sequence, many RNAs (cRNAs) 174 are transcribed by the T7 RNA polymerase. The forward primer 180 hybridizes to the transcribed RNA 174, reverse transcription proceeds, and whereby cDNA 175 is synthesized. Further, the primer 181 for introduction hybridizes to cDNA 175, a DNA strand is synthesized, and a double-stranded cDNA 176 is newly synthesized.

A Molecular Beacon probe 185 hybridizes to a sequence that is complementary to the first sequence portion 183 that has been introduced to the double-stranded cDNA 176. As a result of this hybridization, a loop structure of the Molecular Beacon probe 185 is dissociated, energy transfer caused by a quencher is eliminated, and the Molecular Beacon probe 185 emits fluorescence.

As shown in FIG. 12, the Molecular Beacon probe 185 is constituted by a sequence portion 190 that hybridizes to the target RNA and a sequence portion 191 that forms a stem structure. When the target RNA does not exist, a loop structure of the probe is maintained and fluorescence is not emitted due to energy transfer. When the target RNA exists, a loop structure of the probe is dissociated and a distance between a fluorophore and a quencher becomes larger, and whereby fluorescence is emitted due to elimination of energy transfer.

Similarly with the case shown in FIG. 12, the forward primer may comprise the first sequence portion 54 or 74 located at a position closer to the 5′ end of a sequence that hybridizes to its target gene.

As described above, the probe 15 hybridizes to the second strand cDNA 3 that is generated during the NASBA® reaction, is hydrolyzed by exonuclease or ribonuclease H, and emits fluorescence. Accordingly, the amount of emitted fluorescence is increased depending on the amount of the amplified target genes. Thus, the amount of the target genes contained in the sample can be assayed. Unlike PCR, the NASBA® reaction proceeds under isothermal conditions (40° C. to 42° C.). Thus, amplified by-products derived from genomic DNA can be reduced. This can improve accuracy of detecting the target genes.

### 5. Simultaneous Detection of Several Target Nucleic Acids

A method for simultaneously detecting several target genes (simultaneous detection of several target nucleic acids) using two or more types of universal probes and using a single reaction vessel for one target gene is described.

5.1 Design of Two or More Types of Universal Probes

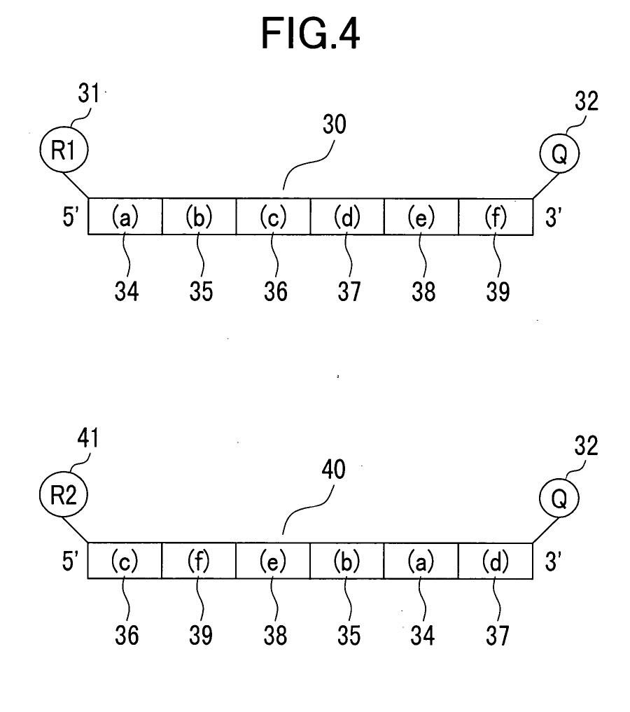

FIG. 4 schematically shows structures of two types of universal probes, probe A (30 in FIG. 4) and probe B (40 in FIG. 4), which are used when simultaneously detecting several target nucleic acids.

Probe A is labeled at its 5′ end with a fluorophore 31 indicated as “R1” in the drawing and at its 3′ end with a quencher 32, which quenches the R1-derived fluorescence through fluorescent energy transfer. As with the case of probe A, probe B is labeled at its 5′ end with the fluorophore 41 indicated as “R2” in the drawing and at its 3′ end with the quencher 32 for quenching fluorescence.

R2 that labels the 5′ end of probe B should be a fluorophore that emits light at a fluorescent wavelength different from that of R1. Thus, whether or not the fluorescence is derived from probe A or probe B can be determined based on the different fluorescent wavelengths between R1 and R2.

Further, probe A and probe B should be designed to hybridize to each target gene with the same reaction properties. As shown in FIG. 4, the sequence of probe A and that of probe B are each constituted by module sequences 34 to 39 each consisting of 3 or 4 bases. The number of module sequences that constitute the probe is not particularly limited. In general, it is preferably about 5 to 8. Both terminal bases of each module sequence are the same. The sequence of probe B is constituted by rearranging the order of modules having terminal bases identical as compared with the sequence of probe A. Since modules having the same terminal bases are rearranged, the base sequences at the joining portions between modules of probe A are the same as those of probe B. Probe A and probe B are constituted by the same modules. Thus, the thermodynamic properties of probe A are equivalent to those of probe B, and Tm values of probe A and probe B are identical to each other based on the calculation by the nearest neighbor method.

Specifically, the entire sequence of probe A is different from the entire sequence of probe B. However, these probes substantially have the same Tm values, and can hybridize to their complementary sequences with the same reaction properties, and therefore they were allowed to simultaneously react in the same reaction tube. Thus, accurate analysis can be made when these probes are used for quantitative analysis. The design of two types of probes was described above. Three or more types of probes can be similarly designed.

5.2 Synthesis of the Gene to be Analyzed (Reverse Transcription into CDNA)

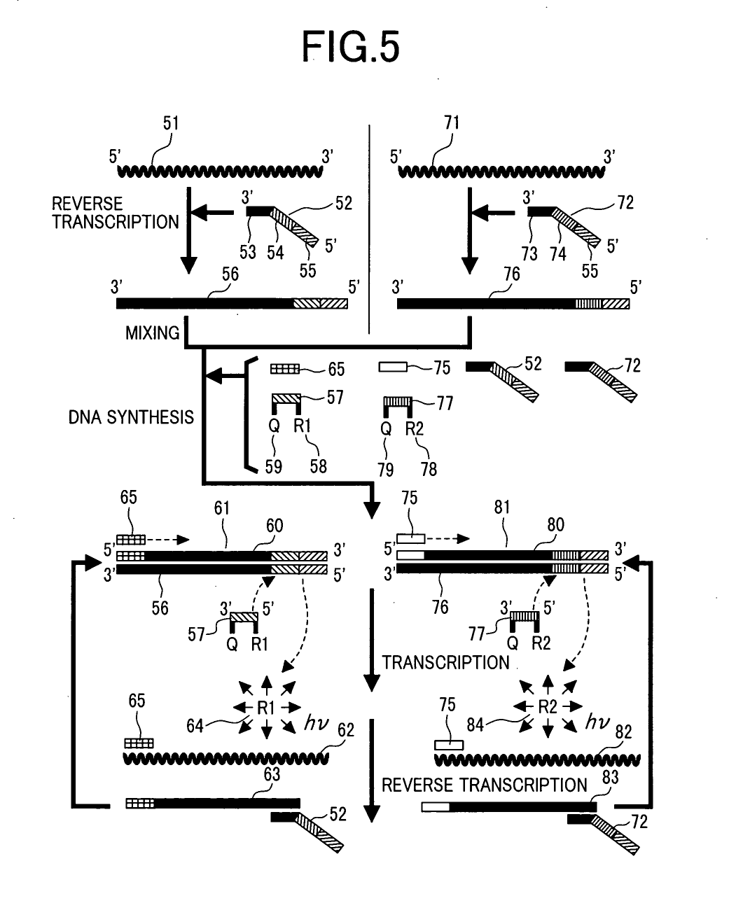

FIG. 5 schematically shows a method for detecting two types of target genes (genes (i) and (ii)) using probe A and probe B by amplifying them by NASBA® in a reaction tube. First, cDNA is prepared from the target gene (i). The reference numeral 51 indicates the target gene (i), and the reference numeral 52 indicates a reverse transcription primer. The reverse transcription primer 52 is constituted by the sequence portion 53 hybridizing to the target gene, the sequence portion 54 located closer to the 5′ end than the sequence portion 53 and consisting of a sequence identical to the probe for detection, and the sequence portion 55 located closer to the 5′ end than the sequence portion 54 and comprising the T7 promoter sequence. Reverse transcription is carried out using this primer in the same manner as in 4. above. Thus, the first strand cDNA 56 in which the sequence portion 54 and the sequence portion 55 have been introduced is obtained.

Another cDNA is synthesized from the target gene (ii) in the same manner as in the preparation of the first strand cDNA 56. The reference numeral 71 indicates the target gene (ii), and the reference numeral 72 indicates a reverse transcription primer. The reverse transcription primer 72 is constituted by the sequence portion 73 hybridizing to the target RNA, the sequence portion 74 located closer to the 5′ end than the sequence portion 53 and consisting of a sequence identical to the probe for detection, and the sequence portion 55 located closer to the 5′ end than the sequence portion 74 and comprising the T7 promoter sequence. Similarly, the target gene (ii) is subjected to reverse transcription to obtain the first strand cDNA 76 comprising the sequence portion 74 and the sequence portion 55 introduced therein. The sequence portion 55 of the reverse transcription primer 52 and the sequence portion 55 of the reverse transcription primer 72 are identical to each other. The sequence portion 54 of the reverse transcription primer 52 and the sequence portion 74 of the reverse transcription primer 72 correspond to probe A and probe B, respectively, as shown in FIG. 4 and they have the same Tm values.

5.3 Simultaneous Detection

Simultaneous detection is schematically described with reference to FIG. 5. A part of the resulting first strand cDNA 56 and a part of the first strand cDNA 76 are taken out of the reaction tube, and equivalent amounts thereof are mixed in a new reaction tube. The resultant is designated as a template. As shown in FIG. 5, the first strand cDNA 56 and the first strand cDNA 76 may be prepared in separate reaction tubes. Alternatively, they may be prepared in the same reaction tube. The forward primer 65 hybridizing to the first strand cDNA 56 and the first strand cDNA 76, the reverse transcription primer 52 (also functions as the primer for introduction), and the reverse transcription primer 72 (also functions as the primer for introduction) are used.

The probe 57, which has the same sequence as the sequence portion 54 introduced into the first strand cDNA 56, and the probe 77, which has the same sequence as the sequence portion 74 introduced into the first strand cDNA 76, are used as universal probes for detection.

The probe 57 is labeled with the fluorophore 58 indicated as “R1” in the drawing and the quencher 59 indicated as “Q” in the drawing. The probe 77 is labeled with the fluorophore 78 indicated as “R2” in the drawing and the quencher 79 indicated as “Q” in the drawing. The quenchers 59 and 79 may be the same if such quenchers can eliminate fluorescence emitted by the fluorophores 58 and 78, respectively. When the probe 57 and the probe 77 are in intact states, fluorescence of the fluorophore 58 and that of the fluorophore 78 are eliminated through fluorescent energy transfer.

The second strand cDNA 60 is synthesized from the first strand cDNA 56 with the aid of a reverse transcriptase. Thus, the double-stranded cDNA 61 having the T7 promoter sequence is generated. The second strand cDNA 80 is synthesized from the first strand cDNA 76. Thus, the double-stranded cDNA 81 having the T7 promoter sequence is generated. Because the double-stranded cDNA 61 and 81 have the T7 promoter sequences, RNA (cRNA) 62 and 82 are transcribed by the T7 RNA polymerase. The forward primer 65 hybridizes to the transcribed RNA 62, reverse transcription proceeds, and cDNA 63 is synthesized. The primer 52 for introduction hybridizes to the cDNA 63, a DNA is synthesized from the primer, and double-stranded cDNA 61 is newly synthesized. Similarly, the forward primer 75 hybridizes to the transcribed RNA 82, reverse transcription proceeds, and cDNA 83 is synthesized. Further, the primer 72 for introduction hybridizes to cDNA 83, a DNA is synthesized from the primer, and double-stranded cDNA 81 is newly synthesized.

The probe 57 hybridizes to the second strand cDNA 60 of the double-stranded cDNA 61. The probe 57 is hydrolyzed by exonuclease. As a result of the hydrolysis, the fluorophore is released from the probe 57, a free fluorophore 64 is generated, and fluorescence is emitted. The probe 77 hybridizes to the second strand cDNA 80 of the double-stranded cDNA 81. The probe 77 is hydrolyzed by exonuclease. As a result of the hydrolysis, the fluorophore is released from the probe 77, a free fluorophore 84 is generated, and fluorescence is emitted.

The quantitative ratio between the free fluorophore 64 and free fluorophore 84 that are generated as the reaction proceeds depends on the quantitative ratio between the cDNA 56 and cDNA 76 that are present in the reaction tube before the reaction proceeds. Thus, comparison of the intensities of fluorescent signals emitted from these two fluorophores enables the assay of the abundance ratio between the cDNA 56 and cDNA 76. Specifically, several target genes can be simultaneously detected.

FIG. 5 shows a case where probes 57 and 77 have the DNA backbones and are hydrolyzed by exonuclease. When probes 57 and 77 have the DNA/RNA chimera backbones, detection can be similarly carried out. In this case, probe 57 or 77 hybridizes to second strand cDNA 60 or 80, and only the resulting RNA strand of the DNA/RNA hybrid (probe) is hydrolyzed by ribonuclease H.

As described above, the method of the present invention enables simultaneous assay of the amounts of several target genes in a sample. In this case, accuracy of detection can be enhanced in the diagnosis of infections since several items for one diagnostic subject can be evaluated in one operation.

### 6. Typing of Base Sequences of Viruses

A method for typing viral genes (virus genotyping) in a single reaction vessel using two or more universal probes is now described.

6.1 Design of Two or More Universal Probes

Two or more probes are designed in accordance with the description in 5.1.

6.2 Synthesis of the Gene to be Analyzed (Reverse Transcription into cDNA)

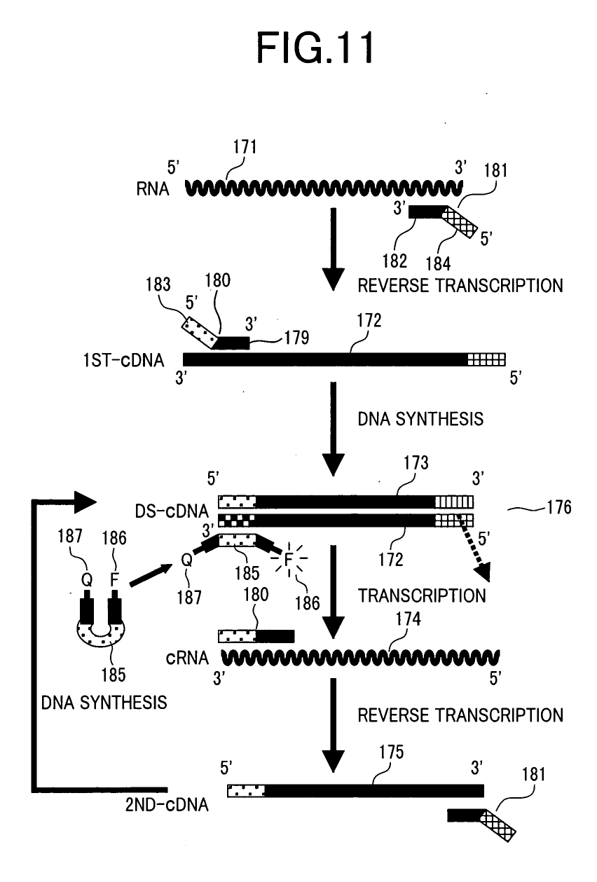

FIG. 9 schematically shows a method for typing base sequences of viruses using probes A, B, C, D, and E in a single reaction tube by NASBA®. FIG. 9 shows a case where the first type among the five genotypes is the target gene. At the outset, cDNA is prepared from the target gene. The reference numeral 101 indicates a target gene, and the reference numerals 112, 122, 132, 142, and 152 indicate reverse transcription primers. The reverse transcription primer 112 is constituted by the sequence portion 113 hybridizing to the target gene, the sequence portion 114 located closer to the 5′ end than the sequence portion 113 and consisting of a sequence identical to the probe for detection, and the sequence portion 115 located closer to the 5′ end than the sequence portion 114 and comprising the T7 promoter sequence. Similarly, reverse transcription primers 122, 132, 142, and 152 are each independently constituted by sequence portions 123, 133, 143, and 153 hybridizing to the target genes, sequence portions 124, 134, 144, and 154 located closer to the 5′ end than the sequence portions 123, 133, 143, and 153 and consisting of a sequence identical to the probe for detection, and the sequence portion 115 located closer to the 5′ end than the sequence portions 124, 134, 144, and 154 and comprising the T7 promoter sequences.

Reverse transcription is carried out in a manner similar to that described in 4. above using these reverse transcription primers to obtain first strand cDNA 102 comprising the sequence portions 114 and 115 introduced therein. As mentioned above, FIG. 9 shows an example wherein the first type of the genes is the target gene. Accordingly, FIG. 9 shows a case where the target gene 101 is allowed to react with the reverse transcription primer 112. When a target gene is of another genotype, this gene is allowed to react with any of the reverse transcription primers 122, 132, 142, or 152. Thus, first strand cDNA comprising sequence portions 124, 134, 144, 154, and 115 introduced therein is obtained. In the process thereafter, when a target gene is of another genotype, a portion that is equivalent to the sequence portion 114 may be substituted with the sequence portions 124, 134, 144, or 154, and a portion that is equivalent to the probe 116 may be substituted with the probes 126, 136, 146, or 156.

The sequence portions 115 of the reverse transcription primers 112, 122, 132, 142, and 152 are identical to each other. The sequence portions 114, 124, 134, 144, and 154 of the reverse transcription primers 112, 122, 132, 142, and 152 have the same Tm values as in the case of probe A and probe B that are shown in FIG. 4.

6.3 Typing in a Single Reaction Vessel

A process of typing in a single reaction vessel is schematically described with reference to FIG. 9. A part or all of the obtained first strand CDNA 102, which is used as a template, is placed in a new reaction tube. Forward primers 111, 121, 131, 141, and 151 hybridizing to first strand cDNA and reverse transcription primers 112, 122, 132, 142,and 152 a reused. One of the forward primers 111, 121, 131, 141,and 151 and one of the reverse transcription primers 112, 122, 132, 142, and 152 are allowed to react with first strand cDNA. FIG. 9 shows an example wherein the forward primer 111 and the reverse transcription primer 112 are allowed to react with first strand cDNA 102.

Probes 116, 126, 136, 146, and 156 are used as universal probes for detection. The universal probes 116, 126, 136, 146, and 156 have sequences identical to the sequence portions 114, 124, 134, 144, and 154 of the reverse transcription primers respectively. In other words, one of the universal probe 116, 126, 136, 146, and 156 has a sequence identical to the sequence portion of the reverse transcription primer that has been introduced in the first strand cDNA. FIG. 9 shows an example wherein the sequence portion 114 has been introduced in the first strand cDNA 102.

The probe 116 is labeled with the fluorophore 117 indicated as “R1” in the drawing and the quencher 108 indicated as “Q” in the drawing. The probe 126 is labeled with the fluorophore 127 indicated as “R2” in the drawing and the quencher 108 indicated as “Q” in the drawing. The probe 136 is labeled with the fluorophore 137 indicated as “R3” in the drawing and the quencher 108 indicated as “Q” in the drawing. The probe 146 is labeled with the fluorophore 147 indicated as “R4” in the drawing and the quencher 108 indicated as “Q” in the drawing. The probe 156 is labeled with the fluorophore 157 indicated as “R5” in the drawing and the quencher 108 indicated as “Q” in the drawing. When the probes 116, 126, 136, 146, and 156 are in intact states, fluorescence of the fluorophores 117, 127, 137, 147, and 157 are eliminated through fluorescent energy transfer.

The second strand cDNA 103 is synthesized from the first strand cDNA 102 with the aid of a reverse transcriptase. This generates the double-stranded cDNA 104 having the T7 promoter sequence. Since the double-stranded CDNA 104 has the T7 promoter sequence, RNA (cRNA) 105 is transcribed with the aid of T7 RNA polymerase. The forward primer 111 hybridizes to the transcribed RNA 105, reverse transcription proceeds, and CDNA 106 is synthesized. Further, the reverse transcription primer 112 hybridizes to cDNA 106, a DNA strand is synthesized from the primer, and the double-stranded cDNA 104 is newly synthesized.

The probe 116 hybridizes to the second strand cDNA 103 of the double-stranded cDNA 104. The probe 116 is hydrolyzed by exonuclease. As a result of the hydrolysis, the fluorophore is released from the probe 116, a free fluorophore 107 is generated, and fluorescence is emitted.

A free fluorophore 107 derived from the probe 116 is generated as the reaction proceeds. A type of a free fluorophore varies since a type of a probe to be digested differs depending on the genotype of the target gene. Thus, inspection of the type and the fluorescent intensity of the fluorophore enables the determination of the genotype of the target gene. Specifically, viruses can be subjected to typing in a single reaction vessel.

As described above, viruses contained in the sample can be subjected to typing in a single reaction tube. This enables the evaluation of several items at a time and direct comparison of types and fluorescent intensities of fluorophores. Thus, typing accuracy can be improved.

### 7. Kit

According to the method of the present invention, the first base sequence and the second base sequence are introduced into a target gene, which are unrelated to base sequences of target genes. This can provide the TaqMan® probe (a universal probe) or the Molecular Beacon (a universal Molecular Beacon) that can be universally used for any kind of target gene. When detecting another target gene, it is sufficient to modify only the sequence of the portion which hybridizes to the target gene of the primer for introduction (the reverse transcription primer) and the sequence of the forward primer.

More specifically, the present invention provides a kit for expressed gene analysis comprising these probes that can be used universally. This kit may be used for detecting a single gene, or it may be used for simultaneously detecting several genes. The characteristics and the constructions of the essential elements of this kit and those of the universal probe are as described above.

In addition to the universal probe as the essential element, the kit of the present invention may comprise other enzymes, reagents, or the like that are necessary for the method for expressed gene analysis of the present invention. Examples thereof include reverse transcriptase, RNA polymerase, ribonuclease H, exonuclease, a buffer for imparting suitable conditions for the enzyme reaction, and other reagents necessary for detecting synthesis products. This kit may comprise the forward primer or the primer for introduction of a specific target gene or may supply a reagent, which is necessary for one reaction, in a fractionated state in a single reaction vessel. The universal probe is not limited to the TaqMan® probe, and it may be the Molecular Beacon.

### EXAMPLES

The present invention is hereafter described in more detail with reference to the following examples, although it is not limited to these examples.

### Example 1

### Detection of Human Papillomavirus Using a Universal Probe

(1) Method of Examination

DNA was extracted as a sample from the cell collected from the uterine cervix, and double-stranded DNA was prepared using the following forward primer and the primer for introduction for amplifying the gene of the human papillomavirus (HPV) E6 protein.

| | |
|---|---|
| Forward primer: | |
| 5′-AAGGG CGTAA CCGAA ATCGG T-3′ | (SEQ ID NO: 1) |
| Primer for introduction: | |
| 5′-AATTC TAATA CGACT CACTA TAGGG CCC | (SEQ ID NO: 2) |
| TTCT CAC TGTT CTC TCAT GTTTG CAGCT | |
| CTGTG CATA-3′ | |

Double-stranded DNA was synthesized by adding 15 pmol of the forward primer (SEQ ID NO: 1), 15 pmol of the primer for introduction (SEQ ID NO: 2), the extracted DNA, and reverse transcriptase (Superscript II reverse transcriptase) to a reaction buffer and then incubating the mixture at 41° C. for 30 minutes.

Amplification and detection by NASBA® were carried out using the Sequence Detection System 7900 (Applied BioSystems). The double-stranded DNA synthesized above, 15 pmol of the forward primer (SEQ ID NO: 1), 15 pmol of the primer for introduction (SEQ ID NO: 2), and 5 pmol of probe (SEQ ID NO: 3) labeled at its 5′ end with a fluorophore FAM and at its 3′ end with a quencher DABCYL were mixed in 20 μl of the reaction solution. Reverse transcriptase, T7 RNA polymerase, T7 gene 6 exonuclease, and substrate dNTP and NTP were added thereto, and the mixture was incubated at 41° C. for 90 minutes. A probe sequence is shown below.

| | |
|---|---|
| Probe: | |
| 5′-(FAM)-CCC TTCT CAC TGTT CTC TCAT- | (SEQ ID NO: 3) |
| (DABCYL)-3′ | |

(2) Results

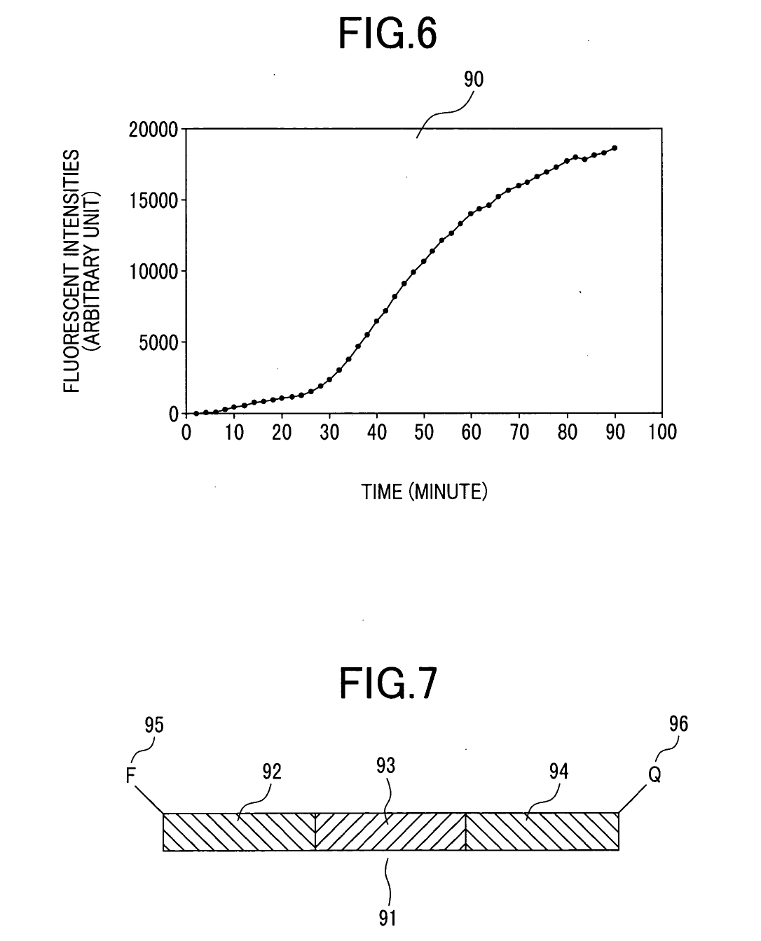

FIG. 6 shows the result of fluorescent detection conducted every given time period. The horizontal axis of the graph represents the reaction time, and the vertical axis thereof represents the fluorescent intensity (arbitrary unit). The graph shows changes in intensities of the fluorescent signal emitted from a sample containing the double-stranded DNA prepared from the target gene. When the amplified product is detected in real time and the level of the fluorescent signal derived therefrom exceeds a certain threshold value, detection of the target gene is confirmed.

### EXAMPLE 2

### Simultaneous Detection of Several Target Nucleic Acids

An example is presented in which several target genes were amplified in a single reaction tube and detected according to the method of the present invention.

(1) Method of Examination

The A chain and the B chain regions of the insulin gene were subjected to reverse transcription, and the first strand cDNAs of these genes were prepared. The forward primer (SEQ ID NO: 4) and the reverse transcription primer (SEQ ID NO: 5) for the A chain region of the insulin gene and the forward primer (SEQ ID NO: 6) and the reverse transcription primer (SEQ ID NO: 7) for the B chain region of the insulin gene are shown below.

| | |
|---|---|
| Forward primer | |
| for the A chain region: | |
| 5′-TGGTG CAGGC AGCCT GCA-3′ | (SEQ ID NO: 4) |
| Reverse transcription | |
| primer for the A chain region: | |
| 5′-AATTC TAATA CGACT CACTA TAGGG CCC | (SEQ ID NO: 5) |
| TTCT CAC TGTT CTC TCAT TAGTT GCAGT | |
| AGTTC TCCAG-3′ | |
| Forward primer | |
| for the B chain region: | |
| 5′-CCAGC CGCAG CCTTT GTGA-3′ | (SEQ ID NO: 6) |
| Reverse transcription | |
| primer for the B chain region: | |
| 5′-AATTC TAATA CGACT CACTA TAGGG CAC | (SEQ ID NO: 7) |
| TCAT CTC TTCT CCC TGTT CAGGT CCTCT | |
| GCCTC CCGG-3′ | |

The reverse transcription primer for the A chain region of the insulin gene and that for the B chain region of the insulin gene were designed in accordance with FIG. 5. Specifically, a sequence portion 55 comprising the T7 promoter sequence existed in common, however, the sequence portion 54 and the sequence portion 74 (underlined portions) each consisting of a sequence identical to the probe for detection located on the 3′ ends were different from each other. Also, the sequence portion 53 and the sequence portion 73 located at the 3′ ends and recognizing each gene region were different from each other.

Equivalent amount of cDNA derived from each obtained sample was taken out of the reaction tube and mixed in a new reaction tube. NASBA® was performed using the resultant mixture as a template, the forward primers (SEQ ID NOs: 4 and 6), the reverse transcription primers (SEQ ID NOs: 5 and 7), and two types of probes.

The following DNA/RNA chimera probes (SEQ ID NOs: 8 and 9) were used as a probe for detecting insulin A chain (probe A) and a probe for detecting insulin B chain (probe B).

| | |
|---|---|
| Probe A: | |
| 5′-(FAM)-d(CCC TTCT)r(CAC UGUU)d(CTC | (SEQ ID NO: 8) |
| TCAT)-(DABCYL)-3′ | |
| Probe B: | |
| 5′-(VIC)-d(CAC TCAT)r(CUC UUCU)d(CCC | (SEQ ID NO: 9) |
| TGTT)-(DABCYL)-3′ | |

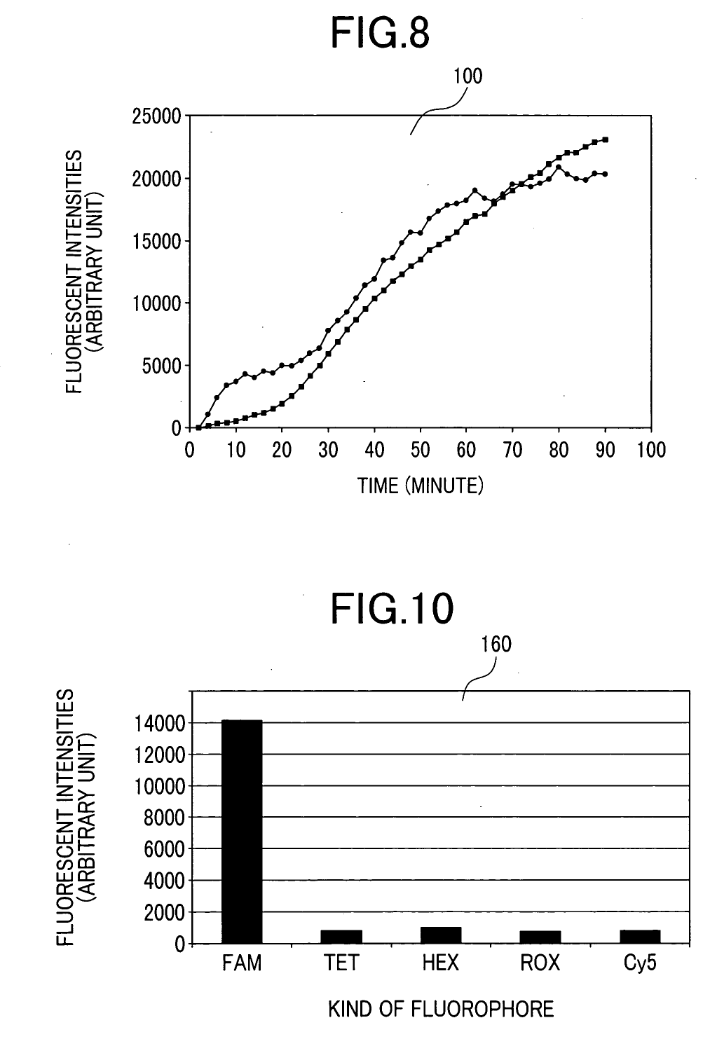

Both probes are designed to have substantially the same Tm values in accordance with FIG. 4. Probe A is labeled at its 5′ end with a fluorophore FAM, probe B is labeled at its 5′ end with a fluorophore VIC, and both probes are labeled at their 3′ ends with quenchers DABCYL, respectively. In both probe A and probe B, 7 bases in the middle of 21 bases constituting the probes are made up of RNA backbones 93 as shown in FIG. 7. There are DNA backbones 92 and 94 neighboring the 5′ end and the 3′ end of the RNA backbone. The 5′ end is labeled with fluorophore R 95, and the 3′ end is labeled with quencher 96. When hybridizing to the target, therefore, 7 bases in the middle of the probe form a DNA/RNA hybrid, they are digested by ribonuclease H, a free fluorophore is generated, and fluorescence is emitted as shown in FIG. 3.

Amplification and detection by NASBA® were carried out using the Sequence Detection System 7900 (Applied BioSystems). The extracted RNA, 10 pmol of the forward primers (SEQ ID NOs: 4 and 5), 10 pmol of the primers for introduction (SEQ ID NOs: 6 and 7), and 5 pmol of probes labeled at their 5′ ends with fluorophores FAM or VIC and their 3′ ends with quenchers DABCYL (SEQ ID NOs: 8 and 9) were mixed in 20 μl of the reaction solution. Further, reverse transcriptase, T7 RNA polymerase, ribonuclease H, and a substrate were added thereto, and the mixture was incubated at 41° C. for 90 minutes.

(2) Results

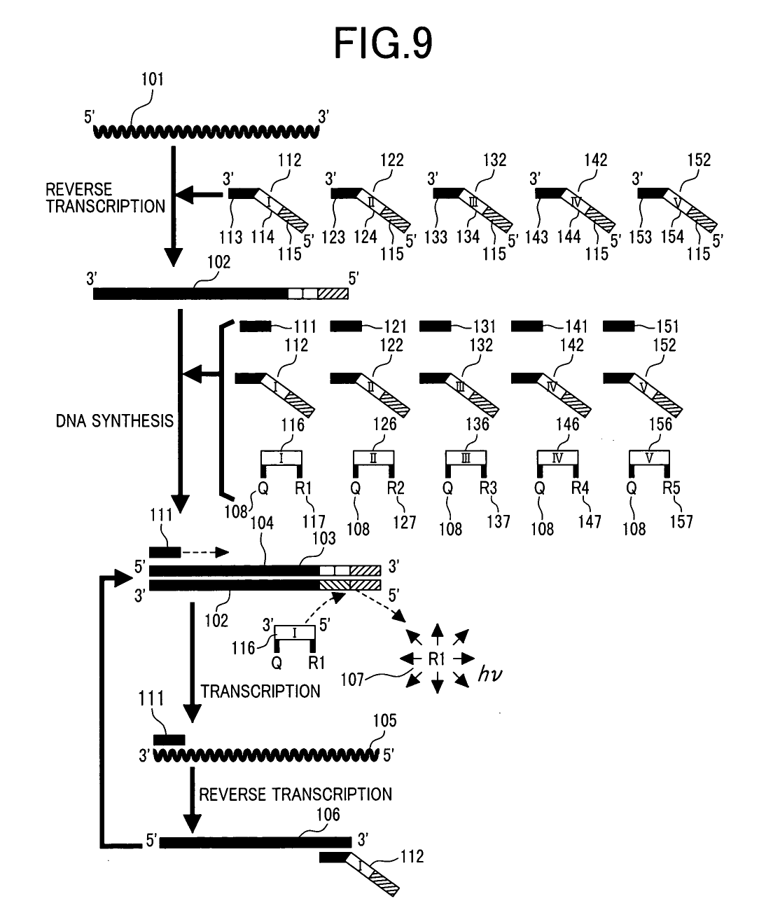

FIG. 8 shows the results of experiments in which the A chain and the B chain regions of the insulin gene were simultaneously detected using two types of probes. The horizontal axis of the graph represents the reaction time, and the vertical axis thereof represents relative fluorescent intensity (arbitrary unit) of a fluorophore. FIG. 8 is a graph showing changes in the intensity of the fluorescent signals when detecting the amplified product of the A chain region of the insulin gene using probe A (indicated by square plots) and when detecting the amplified product of the B chain region of the insulin gene using probe B (indicated by round plots). As is apparent from this graph, probe A and probe B can simultaneously detect their target genes, respectively.

### EXAMPLE 3

### Typing of Base Sequences of Viruses

Subsequently, an example is presented in which amplification and typing of base sequences of viruses are conducted in a single reaction tube in accordance with the method of the present invention. Hepatitis C virus (HCV) is mainly transmitted via the blood, and acute and chronic hepatitis are caused thereby. HCV rapidly causes genetic mutation, and five genotypes, I/1a, II/1b, III/2a, IV/2b, and V/3a, are known as major genotypes. The therapeutic effects of interferon vary depending on these genotypes. Through HCV typing, useful clinical information in terms of pathological conditions or progress thereof can be obtained. Thus, HCV typing is a major item for clinical test.

(1) Method of Examination

The core genes of hepatitis C viruses (HCV) were subjected to reverse transcription to prepare first strand cDNAs of these genes. The following primers were used as the forward primers (SEQ ID NOs: 10 to 14) and the reverse transcription primers (SEQ ID NOs: 15 to 19) for the core genes. RNA extracted from the blood of specimens belongs to any of I/1a, II/1b, III/2a, IV/2b, or V/3a genotype, and FIG. 9 shows RNA of the genotype I/1a.

The reverse transcription primers 112, 122, 132, 142, and 152 for the core genes correspond to genotypes I/1a, II/1b, III/2a, IV/2b, and V/3a, respectively, and they are designed in accordance with FIG. 9. Specifically, the sequence portion 115 comprising the T7 promoter sequence exists in common, however, sequence portions 114, 124, 134, 144, and 154 each consisting of a sequence identical to the sequence of the probe for detection located closer to the 3′ end and sequence portions 113, 123, 133, 143, and 153 located closer to the 3′ end and recognizing each gene region are different from each other.

| | |
|---|---|
| Forward primer | |
| for core gene I/1a: | |
| 5′-GGTCG CAACG TCGAG GTAGA-3′ | (SEQ ID NO: 10) |
| Forward primer | |
| for core gene II/1b: | |
| 5′-CGCAA CCTCG TGGAA GGCGA-3′ | (SEQ ID NO: 11) |
| Forward primer | |
| for core gene III/2a: | |
| 5′-CCCCC CGAGG TTCCC GTGCC-3′ | (SEQ ID NO: 12) |
| Forward primer | |
| for core gene IV/2b: | |
| 5′-CTGTA CGGAA ACGAG GGTTG-3′ | (SEQ ID NO: 13) |
| Forward primer | |
| for core gene V/3a: | |
| 5′-CGACG CGTAA AACTT CTCAA-3′ | (SEQ ID NO: 14) |
| Reverse transcription | |
| primer for genotype I/1a: | |
| 5′-AATTC TAATA CGACT CACTA TAGGG | (SEQ ID NO: 15) |
| CCC TTCT CAC TGTT CTC TCAT GAGCC | |
| ATCCC GCCCA CCAGC-3′ | |
| Reverse transcription | |
| primer for genotype II/1b: | |
| 5′-AATTC TAATA CGACT CACTA TAGGG | (SEQ ID NO: 16) |
| CAC TCAT CTC TTCT CCC TGTT GAGCC | |
| ATCCT GYCCA CGCYA-3′ | |
| Reverse transcription | |
| primer for genotype III/2a: | |
| 5′-AATTC TAATA CGACT CACTA TAGGG | (SEQ ID NO: 17) |
| CTC TGTT CCC TCAT CAC TTCT CCTTA | |
| CCCAC GTTGC GCTAC-3′ | |
| Reverse transcription | |
| primer for genotype IV/2b: | |
| 5′-AATTC TAATA CGACT CACTA TAGGG | (SEQ ID NO: 18) |
| CCC TTCT CTC TCAT CAC TGTT GGTCG | |
| GTGGG GCCCC AATTA-3′ | |
| Reverse transcription | |
| primer for genotype V/3a: | |
| 5′-AATTC TAATA CGACT CACTA TAGGG | (SEQ ID NO: 19) |
| CAC TCAT CCC TGTT CTC TTCT AGGAC | |
| CGGCC TTCGC TCCGA-3′ | |

NASBA® was conducted using a part of the obtained cDNA as a template and forward primers 111, 121, 131, 141, and 151 (SEQ ID NOs: 10,11,12,13, and 14), reverse transcription primers 112, 122, 132, 142, and 152 (SEQ ID NOs: 15,16,17,18, and 19), and 5 types of probes for detecting core genes 116, 126, 136, 146, and 156 (SEQ ID NOs: 20,21,22,23, and 24).

The following primers (SEQ ID NOs: 20, 21, 22, 23, and 24) were used as probes 116, 126, 136, 146, and 156 for detecting core genes (probes A, B, C, D, and E).

| | |
|---|---|
| Probe A: | |
| 5′-(FAM)-GGG TTCT CAC TGTT CTC TCA | (SEQ ID NO: 20) |
| T-(DABCYL)-3′ | |
| Probe B: | |
| 5′-(TET)-CAC TCAT CTC TTCT CCC TGT | (SEQ ID NO: 21) |
| T-(DABCYL)-3′ | |
| Probe C: | |
| 5′-(HEX)-CTC TGTT CCC TCAT CAC TTC | (SEQ ID NO: 22) |
| T-(DABCYL)-3′ | |
| Probe D: | |
| 5′-(ROX)-CCC TTCT CTC TCAT CAC TGT | (SEQ ID NO: 23) |
| T-(DABCYL)-3′ | |
| Probe E: | |
| 5′-(Cy5)-CAC TCAT CCC TGTT CTC TTC | (SEQ ID NO: 24) |
| T-(DABCYL)-3′ | |

Both probes are designed to have substantially the same Tm values in accordance with FIG. 4. Probes A, B, C, D, and E are labeled at their 5′ ends with fluorophores, FAM, TET, HEX, ROX, and Cy5, and their 3′ ends with quenchers DABCYL, respectively. When hybridizing to the target, therefore, they are digested by T7 gene 6 exonuclease, a free fluorophore is generated, and fluorescence is emitted as shown in FIG. 3.

After the amplification by NASBA®, the fluorescent intensity of the sample was assayed using a fluorophotometer. The extracted RNA, 10 pmol of the forward primers (SEQ ID NOs: 10, 11, 12, 13, and 14), 10 pmol of the reverse transcription primers (SEQ ID NOs: 15, 16, 17, 18, and 19), and 5 pmol of the probes labeled at their 5′ ends with a fluorophore FAM, TET, HEX, ROX, or Cy5 and at their 3′ ends with a quencher DABCYL (SEQ ID NOs: 20, 21, 22, 23, and 24) were mixed in 20 μl of the reaction solution. Further, reverse transcriptase, T7 RNA polymerase, ribonuclease H, T7 gene 6 exonuclease, and a substrate were mixed therewith, and the mixture was incubated at 41° C. for 90 minutes for amplification. The fluorescence intensity of the obtained reaction product was assayed and then subjected to typing.

(2) Results

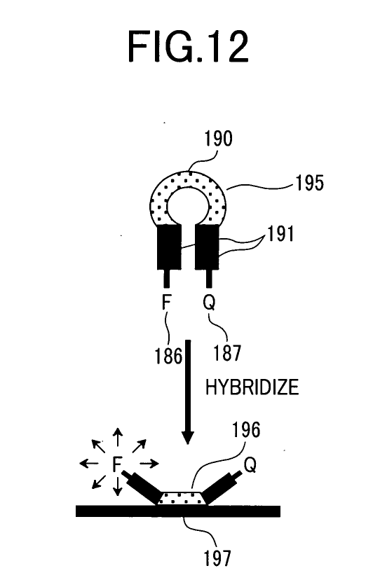

FIG. 10 shows the result of an experiment in which the core genes of the HCV viruses were subjected to typing using 5 types of probes in a single reaction vessel. The horizontal axis of the graph represents a type of each fluorophore, and the vertical axis thereof represents relative fluorescence intensity (arbitrary unit) of the fluorophore. As is apparent from FIG. 10, only the fluorescence intensity of the fluorophore FAM was approximately 10 times greater than those of other 4 types of fluorophores. This means that probe A was digested. Accordingly, it was confirmed that the core gene of the HCV virus was of the genotype I/1a. Thus, typing of core genes can be completed by assaying the types and fluorescence intensities of fluorophores detected in a single reaction vessel.

### Industrial Applicability

The present invention provides a universal probe for expressed gene analysis. Because of its universality, cost-intensive probes do not have to be designed for each use in accordance with the base sequence of the target gene. Also, any type of target genes can be amplified and detected under substantially the same conditions and analysis thereof can be simply conducted. Since amplification is conducted under isothermal conditions, production of by-products can be reduced, and detection can be conducted with high accuracy. The use of two types of universal probes of the present invention in a single reaction vessel enables simultaneous real-time detection of several target genes. This enables more accurate detection and identification of the virus type.

All publications, patents, and patent applications cited herein are incorporated herein by reference in their entirety.

### Free Text of Sequence Listing

SEQ ID NO: 1—Description of artificial sequence: forward DNA primer which is used in NASBA® reaction and hybridizes with Human Papillomavirus DNA

SEQ ID NO: 2—Description of artificial sequence: reverse DNA primer which is used in NASBA® reaction and hybridizes with Human Papillomavirus DNA

SEQ ID NO: 3—Description of artificial sequence: DNA probe which is used in real-time detection of amplified fragments

SEQ ID NO: 4—Description of artificial sequence: forward DNA primer which is used in NASBA® reaction and hybridizes with Human Insulin Gene

SEQ ID NO: 5—Description of artificial sequence: reverse DNA primer which is used in NASBA® reaction and hybridizes with Human Insulin Gene

SEQ ID NO: 6—Description of artificial sequence: forward DNA primer which is used in NASBA® reaction and hybridizes with Human Insulin Gene

SEQ ID NO: 7—Description of artificial sequence: reverse DNA primer which is used in NASBA® reaction and hybridizes with Human Insulin Gene

SEQ ID NO: 8—Description of artificial sequence: DNA/RNA chimera probe which is used in real-time detection of amplified fragments

SEQ ID NO: 9—Description of artificial sequence: DNA/RNA chimera probe which is used in real-time detection of amplified fragments

SEQ ID NO: 10—Description of artificial sequence: forward DNA primer which is used in NASBA® reaction and hybridizes with core gene of hepatitis C virus genotype I/1a

SEQ ID NO: 11—Description of artificial sequence: forward DNA primer which is used in NASBA® reaction and hybridizes with core gene of hepatitis C virus genotype II/1b

SEQ ID NO: 12—Description of artificial sequence: forward DNA primer which is used in NASBA® reaction and hybridizes with core gene of hepatitis C virus genotype III/2a

SEQ ID NO: 13—Description of artificial sequence: forward DNA primer which is used in NASBA® reaction and hybridizes with core gene of hepatitis C virus genotype IV/2b

SEQ ID NO: 14—Description of artificial sequence: forward DNA primer which is used in NASBA® reaction and hybridizes with core gene of hepatitis C virus genotype V/3a

SEQ ID NO: 15—Description of artificial sequence: reverse DNA primer which is used in NASBA® reaction and hybridizes with core gene of hepatitis C virus genotype I/1a

SEQ ID NO: 16—Description of artificial sequence: reverse DNA primer which is used in NASBA® reaction and hybridizes with core gene of hepatitis C virus genotype II/1b

SEQ ID NO: 17—Description of artificial sequence: reverse DNA primer which is used in NASBA® reaction and hybridizes with core gene of hepatitis C virus genotype III/2a

SEQ ID NO: 18—Description of artificial sequence: reverse DNA primer which is used in NASBA® reaction and hybridizes with core gene of hepatitis C virus genotype IV/2b

SEQ ID NO: 19—Description of artificial sequence: reverse DNA primer which is used in NASBA® reaction and hybridizes with core gene of hepatitis C virus genotype V/3a

SEQ ID NO: 20—Description of artificial sequence: DNA probe which is used in detection of amplified fragments

SEQ ID NO: 21—Description of artificial sequence: DNA probe which is used in detection of amplified fragments

SEQ ID NO: 22—Description of artificial sequence: DNA probe which is used in detection of amplified fragments

SEQ ID NO: 23—Description of artificial sequence: DNA probe which is used in detection of amplified fragments

SEQ ID NO: 24—Description of artificial sequence: DNA probe which is used in detection of amplified fragments

We claim:

1. A method for expressed gene analysis comprising: subjecting a gene to be analyzed to nucleic acid amplification using a forward primer specifically hybridizing to the gene to be analyzed, a primer for introduction comprising a first base sequence closer to the 5′ end than a third base sequence comprising a sequence specifically hybridizing to a target gene and comprising a second base sequence closer to the 5′ end than the first base sequence, a probe comprising a base sequence identical or complementary to the first base sequence and labeled at one end with a fluorophore and at another end with a quencher, reverse transcriptase, RNA polymerase, and ribonuclease H and/or exonuclease; digesting the probe bound to the first base sequence by the ribonuclease H or exonuclease at the time of the nucleic acid amplification; and detecting fluorescence emitted by the released fluorophore, thereby assaying the amount of the product of the nucleic acid amplification,
wherein the gene to be analyzed is prepared by introducing the first base sequence and the second base sequence comprising a promoter sequence of RNA polymerase, which are nonspecific to the base sequence of the target gene, into the target gene so that the second base sequence is bound to a position closer to the 5′ end than the first base sequence.

2. The method for expressed gene analysis according to claim 1, wherein the gene to be analyzed is cDNA comprising the first base sequence and the second base sequence introduced therein by subjecting mRNA of the target gene to reverse transcription using a primer for introduction which comprises the first base sequence, which is closer to the 5′ end than the third base sequence comprising a sequence that specifically hybridizes to the target gene and the second base sequence, which is closer to the 5′ end than the first base sequence.

3. The method for expressed gene analysis according to claim 1, wherein the nucleic acid amplification is conducted by sequentially repeating the following steps 1) to 3):
1) transcription of the gene to be analyzed into RNA with the aid of RNA polymerase;
2) reverse transcription of the RNA using the forward primer and the reverse transcriptase or ribonuclease H to synthesize single-stranded cDNA; and
3) synthesis of the gene to be analyzed from the single-stranded cDNA using the primer for introduction and DNA polymerase.

4. The method for expressed gene analysis according to claim 1, wherein the nucleic acid amplification is conducted by sequentially repeating the following steps 1) to 3):
1) transcription of the gene to be analyzed into RNA with the aid of RNA polymerase;
2) reverse transcription of the RNA using the forward primer and the reverse transcriptase or ribonuclease H to synthesize single-stranded cDNA; and
3) synthesis of the gene to be analyzed from the single-stranded cDNA using the primer for introduction and the reverse transcriptase.

5. The method for expressed gene analysis according to claim 1, wherein the nucleic acid amplification is conducted at a substantially single temperature.

6. The method for expressed gene analysis according to claim 5, wherein the single temperature is between 37° C. and 55° C.

7. The method for expressed gene analysis according to claim 1, wherein the RNA polymerase is T7 RNA polymerase and the second base sequence comprises the T7 promoter sequence.

8. The method for expressed gene analysis according to claim 1, wherein two or more target genes are simultaneously detected in a single reaction vessel using two or more types of probes.

9. The method for expressed gene analysis according to claim 8, wherein the melting temperatures (Tm values) of the two or more types of probes are substantially the same.

10. A kit for expressed gene analysis comprising: at least one probe comprising a base sequence identical or complementary to a first base sequence, and labeled at one end with a fluorophore and at another end with a quencher,
wherein both the first base sequence and the second base sequence comprising a promoter sequence of RNA polymerase are nonspecific to a base sequence of a target gene.

11. The kit for expressed gene analysis according to claim 10 comprising two or more types of probes having substantially the same Tm values.

12. The kit for expressed gene analysis according to claim 11, wherein each of the two or more types of probes comprises several module sequences of 3 or 4 bases, both of the terminal bases of each module sequence are identical to each other, and each probe is constituted by rearranging the order of the module sequences having identical terminal bases.

13. The kit for expressed gene analysis according to claim 10, wherein the second base sequence comprises the T7 promoter sequence.
